(19) United States
(12) Patent Application Publication
(10) Pub. No.: US 2006/0064122 A1
(43) Pub. Date: Mar. 23, 2006

(54) Ophthalmological surgical instrument

(75) Inventor: Antoine Sempe, Issy-Les-Moulineaux (FR)

Correspondence Address: YOUNG & THOMPSON, 745 SOUTH 23RD STREET, 2ND FLOOR, ARLINGTON, VA, 22202, US

(73) Assignee: MORIA S.A., ANTONY (FR)

(21) Appl. No.: 11/051,621

(22) Filed: Jan. 27, 2005

(30) Foreign Application Priority Data
Sep. 17, 2004 (FR) 04 09837

(51) Int. Cl. A61F 9/00

(52) U.S. Cl. 606/166

(57) ABSTRACT

An ophthalmological surgical instrument for creating a corneal flap restricted to the epithelial layer of the cornea, which comprises a blade having an active edge that is cylindrical, with its surface being provided with roughnesses.

The present invention relates to an ophthalmological surgical instrument, in particular for refractive surgery.

### BACKGROUND OF THE INVENTION

Vision defects that are due to the cornea can be corrected either by glasses or contact lenses, or else surgically by changing the curvature of the cornea.

Amongst the surgical methods that are known, mention can be made of those which consist in using a microkeratome to form a corneal flap which, on being raised, reveals a stroma bed that is sculpted to the shape that corresponds to the desired correction. The flap is then folded back down onto the bed that has been modified in this way in order to reconstitute the outside surface of the cornea. This modification leads to a different thickness for the cornea, and above all to a different curvature for the surface of the cornea, thereby giving it optical power that is different by an amount that is previously determined for the purpose of obtaining the desired correction effect.

That technique which follows on from a technique of ablating the surface of the cornea (by means of an excimer laser) is not suitable for application under all circumstances, and in particular when the subject for treatment presents a cornea that is too fine.

Another surgical technique that is presently under development consists in removing from the cornea only the epithelial layer in order to sculpt the portion revealed in that way. The sculpted portion becomes naturally covered again by a new epithelium that replaces the epithelium that has been removed. It is believed that that technique presents the advantage of reducing the optical aberrations that are inherent to using a microkeratome, due to the limits reached in mastering the uniformity of the thickness of the corneal flap and in the state of the under-surface of the flap.

That “epithelial” technique leads to slower healing and requires closer follow-up of the treated subject, and appears to be painful for the subject.

Instead of removing purely and simply a portion of the epithelial layer of the cornea by means of a trephine, and applying an alcohol-based solution to assist in peeling off a disk of epithelium, attempts have been made to produce and preserve an epithelial flap. Hand tools have thus been developed for this purpose, even tools of the microkeratome type, implementing a special blade such as that described by document WO 03/092544.

### OBJECT OF THE INVENTION

The present invention seeks to provide another solution to the problem posed by forming an epithelial flap for implementing a corneal surgical technique known as laser epithelial keratomileusis (Lasek).

### BRIEF SUMMARY OF THE INVENTION

To this end, the invention provides an ophthalmological surgical instrument for creating a corneal flap restricted to the epithelial layer of the cornea, the instrument including a blade having an active edge that is cylindrical, with its surface being provided with roughnesses.

In the meaning of the invention, the term “cylindrical” is used to designate a surface constituting the locus of a straight line (a generator line) moving parallel to itself and following an arbitrary curve. Thus, the “cylindrical” surface in the meaning of the invention may be a surface having curvature that is continuous, or a surface having facets.

Like the blade of a microkeratome, the blade is driven with rapid reciprocating motion of small amplitude parallel to the direction of the above-mentioned generator line. The blade thus acts like a rasp which is easily inserted in the cleavage surface that exists in the thickness of the cornea between the layer of epithelial cells and Bowman's membrane, without any risk of injuring Bowman's membrane or penetrating into the stroma body of the cornea, contrary to that which is observed when using a cutter blade with a sharp edge.

Various embodiments for the roughnesses have been envisaged.

In a first case, they comprise microdepressions that intersect the active surface in the form of a plurality of sharp edges of dimensions that are of the same order as the dimensions of an epithelial cell.

The roughnesses may also be constituted by microribs extending perpendicularly to the generator lines of the surface. The dimensions of the roughnesses are assessed relative to the dimensions of epithelial cells. Their dimensions should lie in the range a few tenths of a micrometer to a value of about one-fifth of the mean thickness of the epithelial layer.

Other characteristics and advantages of the invention appear from the description given below of various embodiments of the invention.

### BRIEF DESCRIPTION OF THE DRAWING

Reference is made to the accompanying drawing, in which:

FIG. 1 shows a first embodiment of an active edge of a blade in accordance with the invention; and

FIGS. 2, 3, and 4 show various embodiments of the blade of the invention.

### DETAILED DESCRIPTION OF THE INVENTION

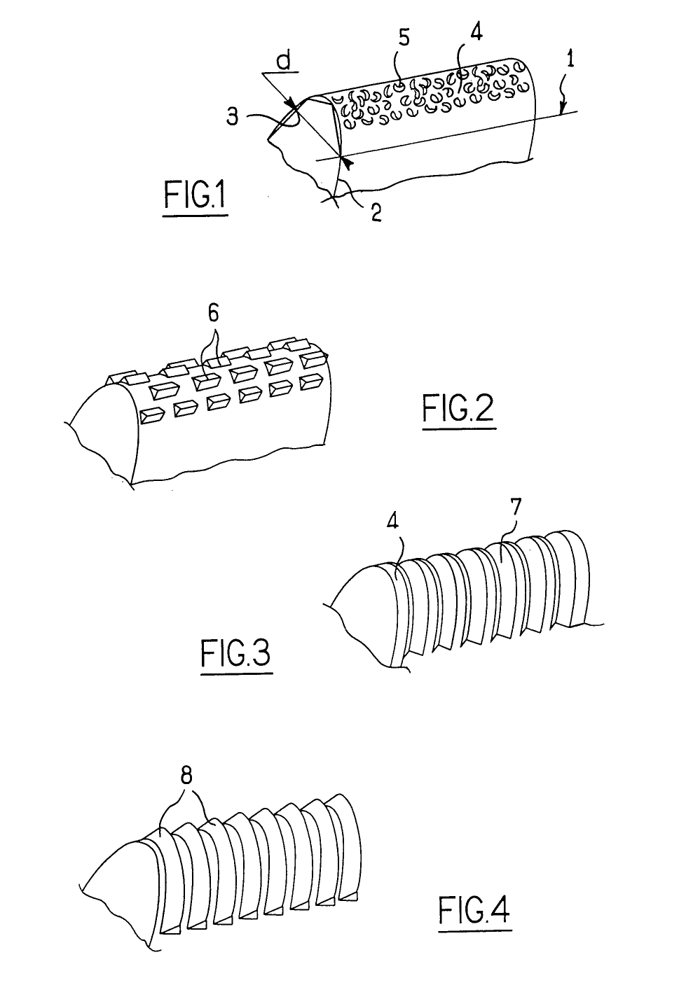

In the figures, only the active portions of blades of the invention are shown, with their general shape being similar to that of the blades used in microkeratomes as shown in numerous documents in the state of the art. The profile of the surface of the active portion of each blade as shown in the figures is convex and substantially parabolic. The surface of this active edge is thus the surface generated by a generator line 1 moving parallel to itself and following a parabolic profile 2. The profile may be a polygonal profile as referenced 3 in FIG. 1. The surface of the active edge is thus a surface that is “cylindrical” in the geometrical meaning of the word.

This figure shows the cylindrical surface 4 provided with a plurality of roughnesses that in this case are implemented in the form of microdepressions 5 which form a multitude of randomly-oriented cutting edges where they intersect the surface 4. The dimensions of these microdepressions 5 and of the cutting edges they define together with the surface 4 are of the same order as the dimensions of the epithelial cells. In addition, the dimension d corresponding to the width of the active portion is of the same order as the thickness of a layer of cells that is about ten cells thick.

In FIG. 2, the roughnesses are constituted by microprojections 6 of dimensions having the same order of magnitude as the depressions 5 in the preceding figure, and obtained by fluting the active surface of the blade edge.

In FIG. 3, the roughnesses are constituted by transverse ribs 7 hollowed out in the surface 4, while in FIG. 4 they are transverse ribs 8 whose top edges lie in said surface 4.

Tests have shown that such a blade makes it possible to produce an epithelial flap of the cornea by sliding and without injuring or perforating Bowman's membrane.

What is claimed is:

1. An ophthalmological surgical instrument for creating a corneal flap restricted to the epithelial layer of the cornea, the instrument comprising a blade having an active edge that is cylindrical, with its surface being provided with roughnesses.

2. An instrument according to claim 1, wherein the roughnesses are formed by microdepressions which form a plurality of cutting edges where they intersect the surface of the active edge, the cutting edges being of dimensions of the same order as the dimensions of an epithelial cell.

3. An instrument according to claim 1, wherein the roughnesses are formed by microribs extending perpendicularly to the generator line of the above-mentioned cylindrical surface.

4. An instrument according to claim 1, wherein the roughnesses are formed by microprojections in relief on the above-mentioned cylindrical surface.
